(19) United States
(12) Patent Application Publication
(10) Pub. No.: US 2020/0128935 A1
(43) Pub. Date: Apr. 30, 2020

(54) Hair dryer with built-in laser diodes

(71) Applicant: Donald Turner, Williamsburg, VA (US)

(72) Inventor: Donald Turner, Williamsburg, VA (US)

(21) Appl. No.: 16/172,793

(22) Filed: Oct. 27, 2018

(51) Int. Cl. A45D 20/10; A61N 5/06; A46B 15/00

(52) U.S. Cl. CPC A45D 20/10; A61N 5/0617; A61N 2005/067; A46B 2200/104; A61N 2005/0652; A46B 15/0002

(57) ABSTRACT

The invention is directed to a hair dryer with built-in laser diodes. The hair dryer provides one or more rows of laser diodes between rows of brush bristles to stimulate hair growth. The hair dryer provides an electric motor which operates quietly, and is battery operated with USB port and ionic technologies designed for the individual user's portability. A charging cradle with power cord is provided. The hair dryer also includes an air ionizer and a large assortment of attachments for additional grooming. Electric toothbrush and hair trimmer attachments may also be provided.

This application relates back to, and claims the priority of Provisional Patent Application No. 62/582,790 filed on Nov. 7, 2017.

### STATEMENT REGARDING FEDERALLY SPONSORED RESEARCH OR DEVELOPMENT

Not Applicable

### PARTIES TO A JOINT RESEARCH AGREEMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The invention relates generally to hair care accessories, and in particular to hair dryer with built-in laser diodes. In recent years, much progress and many innovations have been made regarding hair growth with laser technologies. More and more people are seeking ways of growing healthier hair and more beautiful without chemicals or surgery from implants. Before now there has not been a combination of laser technologies and a hair dryer in one invention. Most users must multi-task throughout the day, such as communicating on the telephone while wanting to watch a favorite show on television, therefore desirable added features to the hair dryer's designed are to operate very quietly which will allow the user to multi-task if needed, and with much more easily with its balance and ergonomic design.

A search of the prior art reveals various devices which have been developed to address this set of problems, or related problems. None are closely related to the present invention, but several include features which resemble those of the present invention. Each has proven to be less than satisfactory for the present purpose in its own way.

Hair dryer having a laser emission function, South Korean Patent No. KR200369961Y1 (priority Sep. 9, 2004), provides a hair dryer which emits far-infrared radiation which is generated in accordance with the low-output laser to the scalp or skin of the body, and prevent hair loss. The hair dryer has a laser emission ability to alleviate pain is disclosed. Hair dryer provides an inlet to which an external air inlet at one side and formed with a discharge port in an air-discharge inlet to the other, forming a body having a handle on the outer peripheral surface and a. The main body is provided with a drive motor and the heater element having the air blowing fan are installed respectively to heat while discharging the incoming air. The handle, the operating switch is provided to an external power source to the driving motor and the heater elements and stopping. Body is connected to the operation switch is provided with a laser light for emitting a low-power laser. Thus hair dryer having a laser emission function is configured, hair loss by stimulating the scalp metabolism and to infiltrate the far-infrared rays generated when emitting a laser to the scalp or skin of the human body stimulate the scalp, the hair follicles with the hot air and induces the growth of the hair to prevent the other hand it is possible to relieve pain through telangiectasia skin, promoting blood circulation and muscle relaxation.

Laser hair dryer anion for hair growth, Chinese Patent No. CN207767748U (priority Nov. 7, 2017), provides a laser anion generating hair dryer, including barrel, the barrel provided at the front end of the blowing nozzle, provided at a rear end of the barrel inlet, screened cap, and a handle fixedly connected to the barrel, the laser further comprising an LED lamp, a laser LED lamp is fixed and disposed within the barrel mouth near the hair. The advantage of the laser is a laser LED lamp capable of generating negative ions and special wavelength, a laser having a wavelength and special anion promoting hair follicle activation, follicular germinal recovery function; small size, hair protective function.

Hair dryer, South Korean Patent No. KR0115654Y1 (priority Sep. 28, 1995), provides a hair dryer that used to after cosmetic beauty salon drying the hair, in particular provided with a far infrared heater in the body of the hair dryer and install the fan so as to blow the air heated in the heater dryer section the drying portion which is connected with a body portion, a laser diode capable of forming a hot-air injection port so as to inject the air to the hair round and oscillating a laser between the hot air injection port circuit with the substrate for injecting from the blowing fan fixed installation, in a cosmetic hair dryers for help accelerate the drying section at the bottom circumference giving make a stand damaged hair because when the hair dry by installing a far-infrared light bulbs by oscillating the laser with the far-infrared hot air drying your hair into healthy hair, hair It relates.

Hair treatment device using laser, South Korean Patent No. KR101155710B1 (priority Nov. 28, 2011), provides a laser hair treatment device intended to prevent damage to hair, maintain healthy hair, and to save energy by using thermoelectric elements. CONSTITUTION: A laser hair treatment device comprises an outlet(10), a laser fixing unit(30), a laser generator(40), a thermoelectric element(50), a heat sink(55), a main housing(70), and a second housing(72). The outlet is formed in a pipe shape. The length of the lower part of the outlet is shorter than that of the upper part of the outlet. The laser fixing unit is installed in one side end of the main housing. The laser fixing unit has a ring shape in which a plurality of fixed holes is equipped. The thermoelectric element is installed to the upper end of the main housing and is mounted in the border of a main housing and a second housing. The heat sink is attached to both sides of the thermoelectric element. In the main housing, a space is formed. The second housing is equipped in one side of the main housing.

Hair dryer capable of preventing hair loss, Chinese Patent No. CN107334249A (priority Apr. 28, 2016), provides a hair dryer capable of preventing hair loss, relating to the field of household appliances. The hair dryer comprises a hair dryer body, wherein an illumination light source is arranged at an air outlet of the hair dry body, and the illumination light source is infrared rays. Preferably, a shell of the hair dryer body is further provided with a control button for the illumination light source. The hair dryer provided by the invention has the beneficial effects that the device with the illumination light source is adopted, after frequent hair blowing, the effects of increasing hair and preventing hair loss can be achieved, and thus the hair dryer has a very good market application valve. Particularly, according to the other technical schemes of the invention, when the irradiation light source emits infrared laser, the good and obvious effects of increasing hair and preventing hair loss are achieved.

A hair dryer with built-in laser diodes, which operates quietly, provides one or more rows of laser diodes between rows of brush bristles to stimulate hair growth while drying and grooming the hair, or using the laser treatment without the dryer in operation, and provides a broad variety of interchangeable attachments, would resolve these problems.

### SUMMARY OF THE INVENTION

Accordingly, the invention is directed to a hair dryer with built-in laser diodes. The hair dryer provides one or more rows of laser diodes between rows of brush bristles to stimulate hair growth. The hair dryer provides an electric motor which operates quietly, and is battery operated with USB port and ionic technologies designed for the individual user's portability. A charging cradle with power cord is provided. The hair dryer also includes an air ionizer and a large assortment of attachments for additional grooming. Electric toothbrush and hair trimmer attachments may also be provided.

Additional features and advantages of the invention will be set forth in the description which follows, and will be apparent from the description, or may be learned by practice of the invention. The foregoing general description and the following detailed description are exemplary and explanatory and are intended to provide further explanation of the invention.

### BRIEF DESCRIPTION OF THE DRAWINGS

The accompanying drawings are included to provide a further understanding of the invention and are incorporated into and constitute a part of the specification. They illustrate one embodiment of the invention and, together with the description, serve to explain the principles of the invention.

FIG. 1 provides a side view, a front view, a front perspective view, and a rear perspective view of the first exemplary embodiment with the short bristled wand attached, displaying the hair dryer 10, the laser diodes 11, the bristles 12, the main hairbrush wand 12A, the air vents 13, the intake vent 14, the speed control 15, the laser diode on/off button 15A, the ionic on/off button 15B, the temperature control 16, the main on/off button 16A, the indicator lights 17, and the handle 18.

FIG. 2 provides a side view, a front view, a front perspective view, and a rear perspective view of the first exemplary embodiment with the medium bristled wand attached, displaying the hair dryer 10, the laser diodes 11, the bristles 12, the medium bristled wand 12B, the air vents 13, the intake vents 14, the speed control 15, the laser diode on/off button 15A, the ionic on/off button 15B, the temperature control 16, the main on/off button 16A, the indicator lights 17, and the handle 18.

FIG. 3 provides a side view, a front view, a front perspective view, and a rear perspective view of the first exemplary embodiment with the long bristled wand attached, displaying hair dryer 10, the bristles 12, the long bristled wand 12C, the intake vents 14, the laser diode on/off button 15A, the main on/off button 16A, and the handle 18.

FIG. 4 provides a side view, a front view, a front perspective view, and a rear perspective view of the first exemplary embodiment with the high speed drying attachment attached, displaying the hair dryer 10, the intake vents 14, the handle 18, and the high speed drying attachment 19A.

FIG. 5 provides a side view, a front view, a front perspective view, and a rear perspective view of the first exemplary embodiment with the long hair curling attachment attached, displaying the hair dryer 10, the intake vents 14, the handle 18, and the long hair curling attachment 19B.

FIG. 6 provides a side view, a front view, a front perspective view, and a rear perspective view of the first exemplary embodiment with the short hair curling attachment attached, displaying the hair dryer 10, intake vents 14, the handle 18, and the short hair curling attachment 19C.

FIG. 7 is a front perspective view of the first exemplary embodiment in the recharging cradle, displaying the hair dryer 10, the handle 18, the recharging cradle 20, and the power cord 22.

FIG. 8 is a front perspective view of the first exemplary embodiment with the recharging cradle, displaying the hair dryer 10, the main on/off button 16A, the handle 18, the recharging cradle 20, the recharging pins 21, the power cord 22, and the charging indicator light 23.

FIG. 9 is a rear perspective view of the first exemplary embodiment with the power cord attached, displaying the hair dryer 10, the handle 18, and the power cord 22.

FIG. 10 is a bottom perspective view of the first exemplary embodiment with the power cord attached, displaying the hair dryer 10, the handle 18, and the power cord 22.

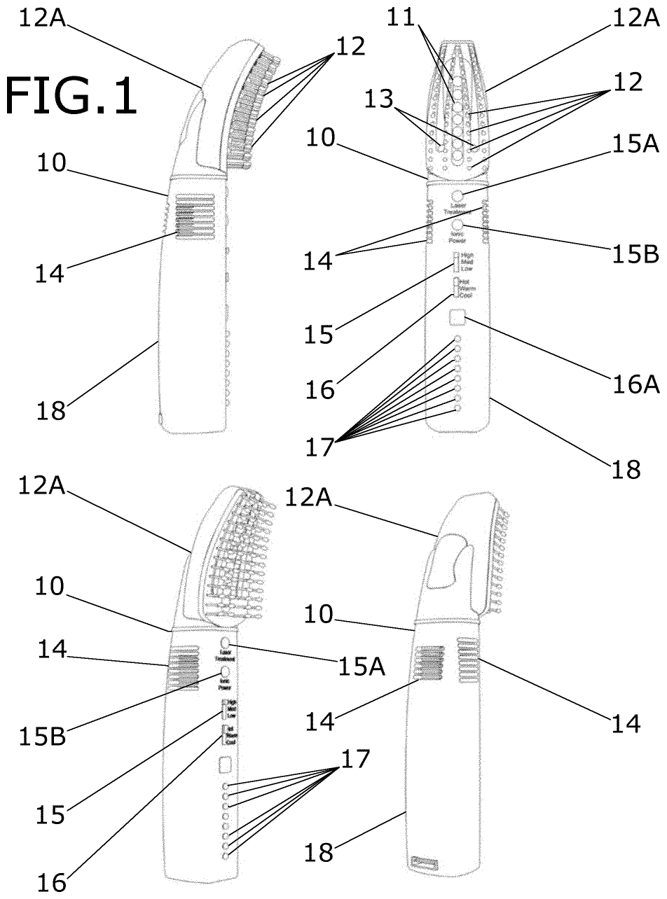

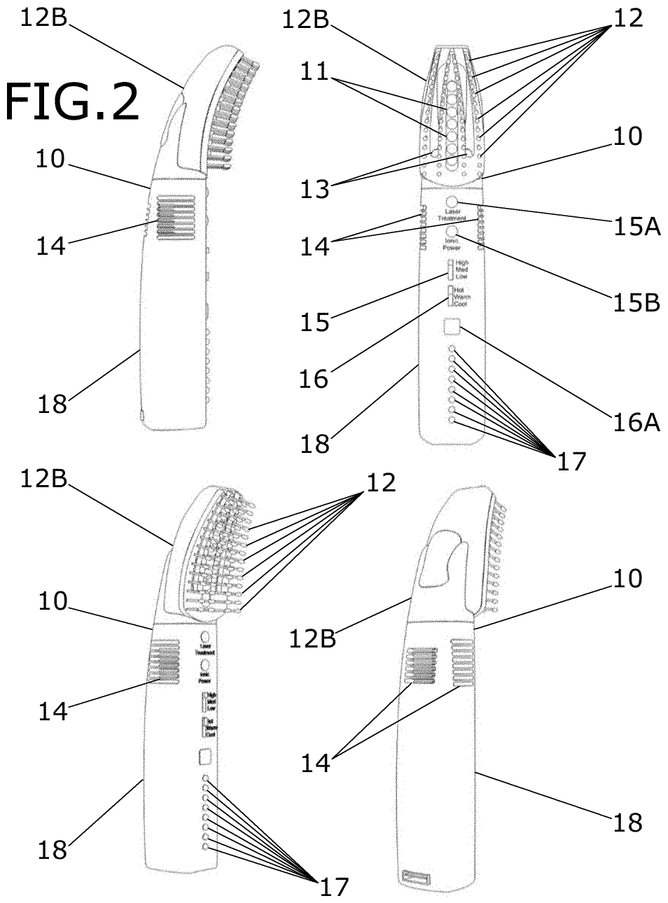

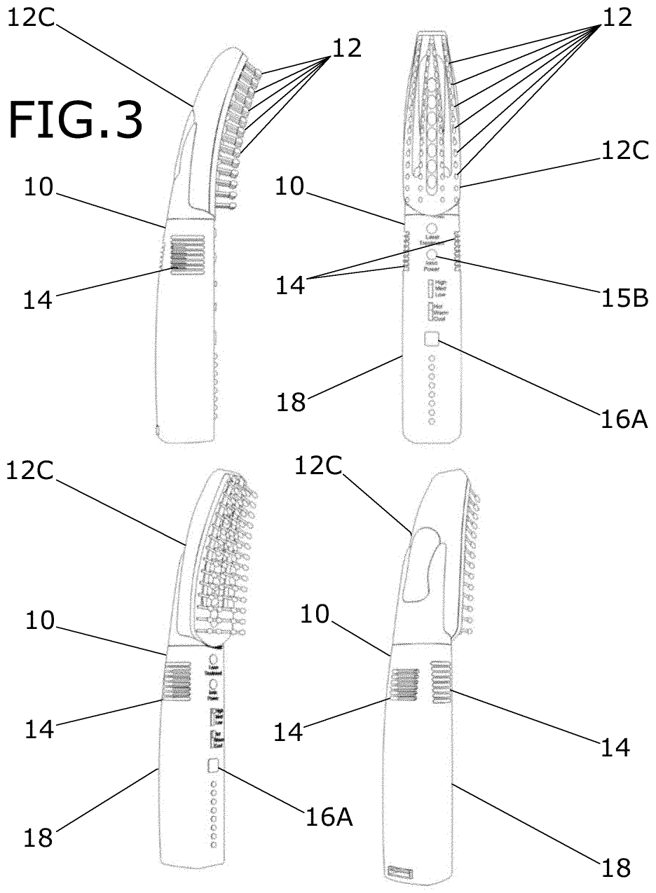

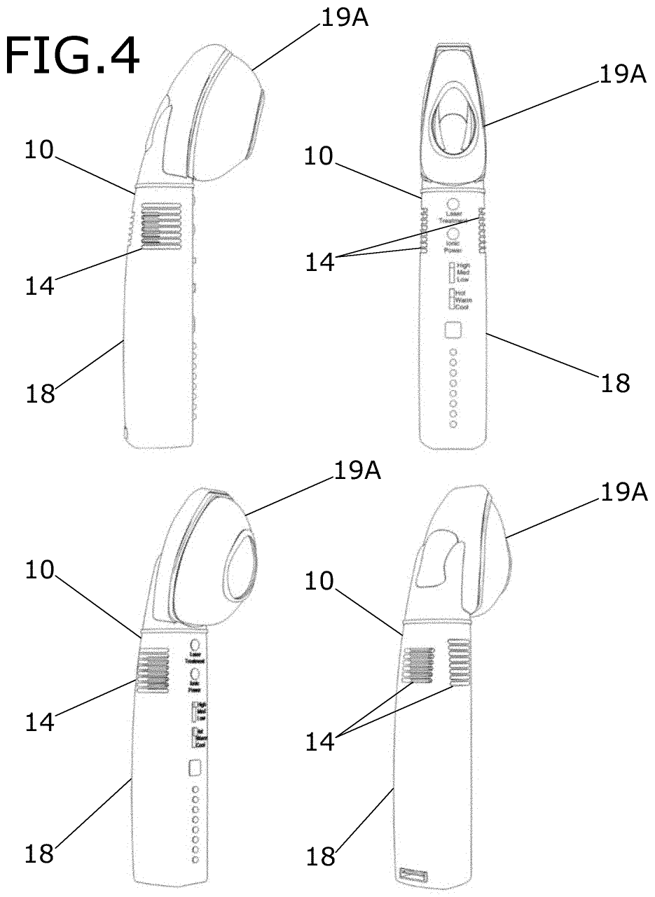

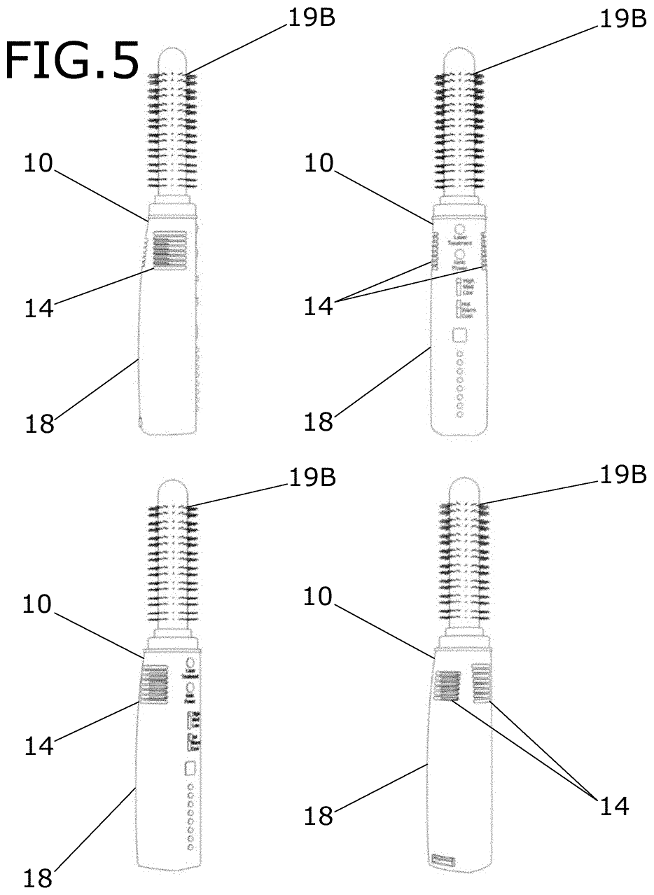

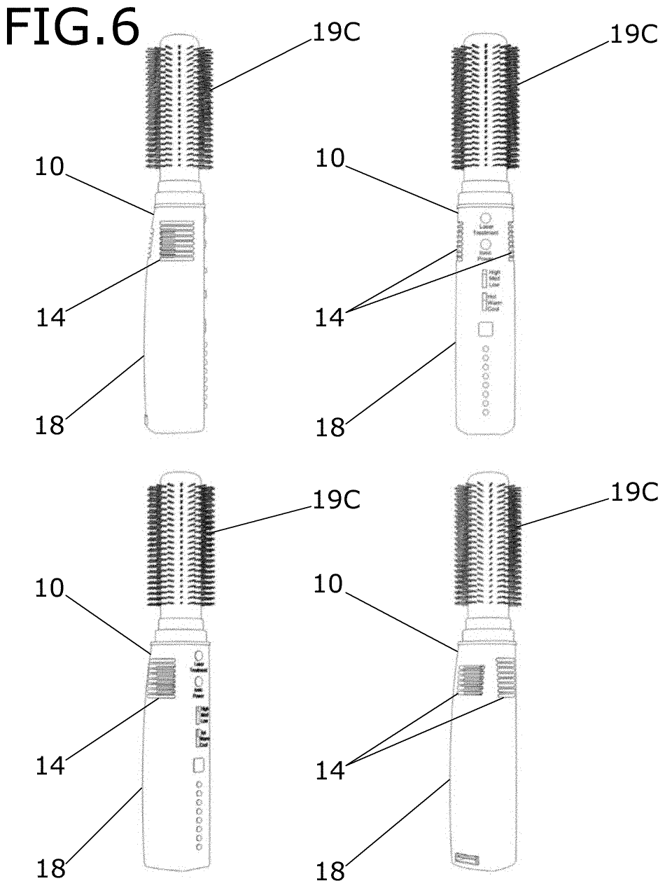

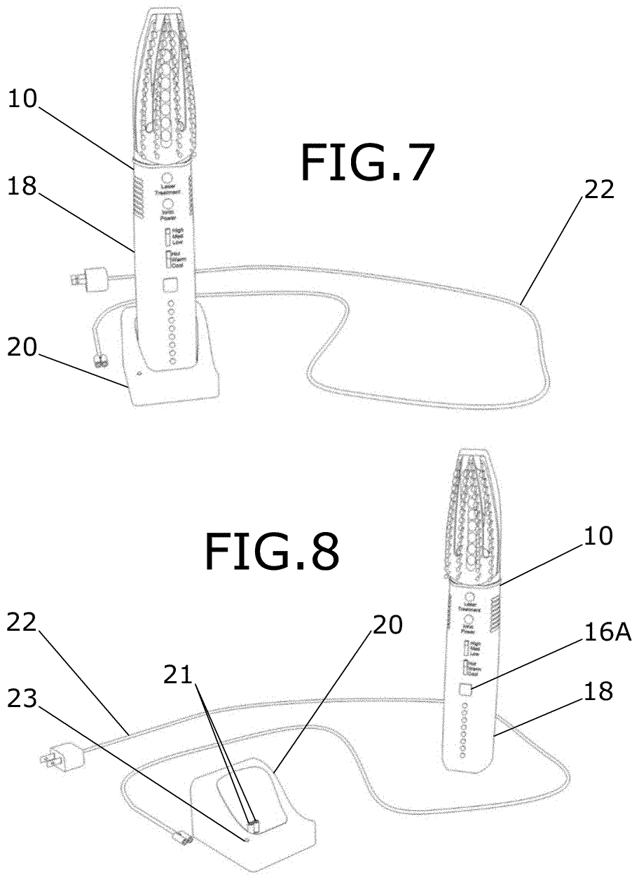

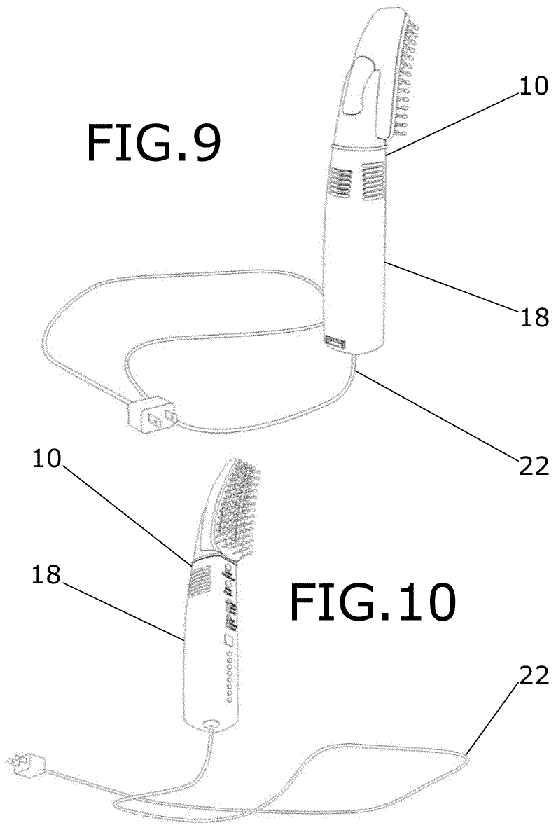

### DETAILED DESCRIPTION OF THE INVENTION

Referring now to the invention in more detail, the invention is a hair dryer 10 with built-in laser diodes 11.

The first exemplary embodiment is comprised of a hair dryer 10 which provides one or more rows of laser diodes 11 between rows of brush bristles 12 to stimulate hair growth. The hair dryer 10 further provides an air ionizer, which is activated with an ionic on/off button 15B. There are two types of ions, negative and positive. Primarily the purpose of the invention is to employ lasers and negative ions.

Negative ions do three things for the hair, and should be used while drying the hair. First, negative ions help smooth and close the cuticles of the hair shaft, which protects the cortex of the hair. By doing this it makes the hair stronger, and allows it to hold more moisture. This allows a person to grow even longer and more beautiful hair with less breakage. Second, negative ions allow the hair to dry much faster by splitting the water molecules up, which in turn would use much less battery power. Third, negative ions enable the hair to produce a shine and luster naturally without chemicals or other treatments.

Positive Ions are principally ozone. The advantage of using ozone technology is that the oxygen kills bacteria around the hair follicle and scalp area and sends much needed oxygen to the hair roots. This promotes hair growth by feeding the follicles with the oxygen.

The hair dryer 10 further provides air vents 13 which expel air to dry the hair, and intake vents 14 to draw air into the motor. A speed control 15 is provided, which is a three-way switch operating options of low, medium, and high speed for the expulsion of air from the air vents 13.

A temperature control 16 is also provided, which is a three-way switch operating options of hot, warm, and cool temperatures for the expulsion of air from the air vents 13. This is a true cooling setting, distinguishing the present invention from prior art which requires the user to keep a button pressed, with a finger or thumb, to keep cool air flowing.

The hair dryer 10 further provides a variety of interchangeable attachments for drying and curling hair, and for other grooming and personal care needs. The attachments preferably include a high speed drying attachment 19A, a long hair curling attachment 19B, and a larger hair curling attachment 19C for use with longer hair.

The hair dryer 10 provides an electric motor which operates quietly, and an internal, rechargeable battery in a handle 18. A plurality of indicator lights 17 on the handle 18 clearly indicate the charge level of the battery. A charging cradle 20 with power cord 22 is provided. The power cord 22 may be plugged into any standard wall outlet. Within the charging cradle 20, two charging pins 23 are provided which may be inserted into corresponding sockets on the lower end of the handle 18 to charge the battery. The charging cradle 20 further provides a charging indicator light 23, which illuminates when the charging cradle 20 is charging the battery.

The hair dryer 10 further provides a main on/off button 16A, which is illuminated in green when activated, and a laser diode on/off button 15A, which is illuminated in red when activated. Thus the hair dryer 10 may be operated with only the laser diodes 11 activated, with the motor activated to dry hair, or both. The hair dryer 10 further provides a USB port, such that the battery may be recharged, and the hair dryer 10 may be operated, while in a vehicle or in other conditions which are less than ideal.

Operating times on a single battery charge, with or without laser diodes being activated, are as follows.

| Fan speed | Heating | Operating time |
|---|---|---|
| Fan low | Cool | Up to 40 plus minutes. |
| Fan mid | Cool | Up to 35 plus minutes. |
| Fan high | Cool | Up to 25 plus minutes. |
| Fan low | Warm | Up to 30 plus minutes. |
| Fan mid | Warm | Up to 25 plus minutes. |
| Fan high | Warm | Up to 20 plus minutes. |
| Fan low | Hot | Up to 15 to 20 minutes. |
| Fan mid to high | Hot | Up to 10 to 15 plus minutes. |
| Laser only | None | Two months or more |

To use the first exemplary embodiment, the user may select and affix the desired attachment to the hair dryer 10, and use the hair dryer 10 to style the user's hair, adjusting the speed control 15 and the temperature control 16 as desired, and activating and deactivating the laser diodes 11 as desired. Negative ions may be generated by clicking on the ionic on/off button 15B. Positive Ions may be generated by double clicking on the ionic on/off button 15B.

The hair dryer 10, the laser diodes 11, the air vents 13, the intake vents 14, the speed control 15, the temperature control 16, the indicator lights 17, the handle 18, the high speed drying attachment 19A, the charging cradle 20, the charging pins 21, and the charging indicator light 23 are preferably manufactured from rigid, durable materials such as plastic, brass, steel, copper alloy, and aluminum alloy.

The brush bristles 12, the long hair curling attachment 19B, and the larger curling attachment 19C are preferably manufactured from a semi-rigid, durable material such as nylon or hog bristles. The power cord 22 is preferably manufactured from braided copper alloy wire sheathed in plastic. Components, component sizes, and materials listed above are preferable, but artisans will recognize that alternate components and materials could be selected without altering the scope of the invention.

While the foregoing written description of the invention enables one of ordinary skill to make and use what is presently considered to be the best mode thereof, those of ordinary skill in the art will understand and appreciate the existence of variations, combinations, and equivalents of the specific embodiment, method, and examples herein. The invention should, therefore, not be limited by the above described embodiment, method, and examples, but by all embodiments and methods within the scope and spirit of the invention.

I claim:

1. A hair dryer providing a row of laser diodes between rows of brush bristles to stimulate hair growth, and further providing an air ionizer, which is activated with an ionic on/off button.

2. The hair dryer of claim 1, wherein the hair dryer further provides air vents which expel air to dry the hair, and intake vents to draw air into the motor.

3. The hair dryer of claim 1, wherein the hair dryer further provides a speed control which is a three-way switch operating options of low, medium, and high speed for the expulsion of air from the air vents.

4. The hair dryer of claim 1, wherein the hair dryer further provides a temperature control, which is a three-way switch operating options of hot, warm, and cool temperatures for the expulsion of air from the air vents.

5. The hair dryer of claim 1, wherein the hair dryer further provides a variety of interchangeable attachments for drying and curling hair, and for other grooming and personal care needs, preferably including a high speed drying attachment, a long hair curling attachment, and a larger hair curling attachment for use with longer hair.

6. The hair dryer of claim 1, wherein the hair dryer further provides an electric motor which operates quietly, an internal, rechargeable battery in a handle, and a plurality of indicator lights on the handle which clearly indicate the charge level of the battery.

7. The hair dryer of claim 1, wherein the hair dryer further provides a charging cradle with power cord which may be plugged into any standard wall outlet.

8. The hair dryer of claim 7, wherein within the charging cradle, two charging pins are provided which may be inserted into corresponding sockets on the lower end of the handle to charge the battery.

9. The hair dryer of claim 7, wherein the charging cradle further provides a charging indicator light, which illuminates when the charging cradle is charging the battery.
